(19) United States
(12) Patent Application Publication
(10) Pub. No.: US 2025/0195150 A1
(43) Pub. Date: Jun. 19, 2025

(54) INSTRUMENT TRACKING AND INSTRUMENT INSPECTION FOR AUTOMATICALLY IDENTIFYING MEDICAL/ SURGICAL ITEMS

(71) Applicant: Aesculap AG, Tuttlingen (DE)

(72) Inventors: Arne Jansen-Troy, Stockach (DE); Dennis Goerz, Tuttlingen (DE); Allan Maas, Konstanz (DE); Matthias Henke, Villingen-Schwenningen (DE); Daniel Kiessling, Villingen-Schwenningen (DE)

(21) Appl. No.: 18/847,315

(22) PCT Filed: Mar. 14, 2023
(86) PCT No.: PCT/EP2023/056517

§ 371 Date: Sep. 16, 2024

(30) Foreign Application Priority Data
Mar. 17, 2022 (DE) 10 2022 106 281.3

(51) Int. Cl. A61B 34/20; A61B 90/00; A61B 90/90; G21F 3/00

(52) U.S. Cl. CPC A61B 34/20; A61B 90/90; G21F 3/00; A61B 2034/2065; A61B 2090/0805

(57) ABSTRACT

A device for automatically identifying medical and/or surgical items includes a capturing apparatus that has and/or uses at least one scanner unit and a detector such that the medical and/or surgical items are located between the scanner unit and the detector at least briefly during automated identification. An imaging and computing unit is preferably located on/in a housing. The at least one scanner unit is a source of electromagnetic radiation. The imaging and computing unit is configured to detect and to output, preferably postoperatively and/or intraoperatively, the completeness of the medical and/or surgical items and/or the absence of at least one medical and/or surgical item. The device can be part of an identification system.

### CROSS-REFERENCE TO RELATED APPLICATIONS

This application is the United States national stage entry of International Application Number PCT/EP2023/056517, filed on Mar. 14, 2023, and claims priority to German Application No. 10 2022 106 281.3, filed on Mar. 17, 2022. The contents of International Application Number PCT/EP2023/056517 and German Application No. 10 2022 106 281.3 are incorporated by reference herein in their entireties.

### FIELD

The present disclosure relates to a device for automatically identifying medical and/or surgical items with a capturing apparatus which comprises at least a scanner unit and a detector.

### BACKGROUND

In the healthcare sector, the trend is increasingly moving toward individual instrument tracking in order to be able to track the respective medical and/or surgical items at instrument OR material level, in particular to be able to check the completeness of the items both postoperatively and intraoperatively. Such completeness checks also serve to reduce large and cost-intensive wastage of medical and/or surgical items and to increase patient safety.

The aspect of patient safety is becoming increasingly important in view of the fact that sometimes around 100 pressure rolls and 40 compresses are used in addition to the other instruments during an emergency operation. Furthermore, hospital statistics show that an average of 3,000 cases occur each year in which foreign bodies such as cloths, swabs or similar items remain in the patient's body after surgical procedures.

In addition to traditional manual methods such as sorting and counting, a wide range of technologies is now being used to support the capturing of surgical instruments or to even fully automate the process.

The state of the art for tracking surgical instruments includes RFID technology, other radio systems such as Bluetooth and optical machine-reading systems such as barcodes, data matrix codes etc. and classic human-readable alphanumeric data as well as systems that use combined technologies.

Examples of RFID technology are disclosed in PCT/EP2017/062129, PCT/GB2015/052977, PCT/IB2020/055731, PCT/US2012/030046. The use of Bluetooth is described, for example, in PCT/CN2014/095452.

Artificial intelligence (AI)-based tracking technologies, such as video and laser scanner systems, are currently becoming established for recognizing, classifying and assigning objects. Developers of self-driving vehicles and robotic industrial systems are pioneers in this segment. AI-based radiological systems are also known for ultrasound and fluoroscopic imaging procedures for optimizing diagnoses. Tracking systems are also used intraoperatively to capture the position of previously defined instruments or their end effectors.

Using RFID technology to read out data shows that usually not 100% of all instruments can be captured and that technical and regulatory requirements also make it difficult to apply RFID tags to existing instruments.

Using barcodes or data matrix codes to read out data can make it much easier to enter or assign article numbers, but currently cannot be executed fully automatically. Low-contrast, corroded or scratched codes also make reading in or out difficult or sometimes impossible.

Optical methods for recognizing objects are already being used to identify or track surgical instruments, wherein these are held in front of a camera, in some cases stereo cameras or 3D camera systems, from different angles and these objects are learned with the help of an AI so that they can be automatically recognized later. In the case of surgical instruments, these are placed individually on an optically homogeneous surface and the lighting conditions are kept constant. In this way, the objects can be recognized—even different, deviating geometries and sizes can be differentiated and assigned to a stored article number.

This optical technology reaches its limits as soon as the lighting conditions change significantly or reflections occur; in these cases, the objects can no longer be clearly assigned. In particular, if many instruments are placed chaotically on top of each other, the system can no longer differentiate between the instruments. Tests have also shown that instruments placed in a bowl, for example, can no longer be clearly recognized or assigned due to the changed optical conditions, in particular reflections and weak contrasts.

Another type of inspection device focuses on the quality inspection of manufactured electronic components, for example to check solder joints and through-hole plating inside circuit boards. Here, image resolutions of 500 nm can be achieved with 3D CT scanners, usually using the laminography method. Topographical properties can thus be visualized. In contrast to the X-ray inspection devices used in the food industry, the test specimens remain in the scanner for a long time without changing position.

### SUMMARY

Against this background, the objective technical object of the present disclosure is to provide a device which eliminates or at least improves the above disadvantages.

Accordingly, the present disclosure relates to a device for automatically, preferably simultaneously, identifying a number, preferably a plurality and further preferably different, medical and/or surgical items, comprising a capturing apparatus, which comprises or uses at least one scanner unit and a detector and/or uses them such that medical and/or surgical items are at least briefly located between the scanner unit and the detector during automatic identification, and an imaging and computing unit preferably arranged on/in a housing, wherein the at least one scanner unit is or has an X-ray source and the imaging and computing unit is provided and configured to recognize and output the completeness of the medical and/or surgical items and/or the absence of at least one medical and/or surgical item, preferably postoperatively and/or intraoperatively, and/or to recognize medical and/or surgical items not only at article number level but also at batch level.

In other words, the present disclosure is based on analogous hardware of known X-ray inspection devices, in particular those systems which have a scanner unit, preferably a line scanner with a conveyor belt for the goods to be inspected. As a rule, the scanner unit is configured as an X-ray source with an imaging unit and a computing unit with corresponding software for imaging.

In yet other words, using X-rays for imaging in combination with software, chaotically arranged medical and/or surgical items can be individually identified and thus tracked/followed. This in turn means that the completeness of the items can be checked immediately postoperatively and also intraoperatively and, if applicable, the absence of surgical instruments can be automatically indicated via a human-machine interface. In addition to increased patient safety, this may also provide improved information on lost items/instruments at a very early stage when they are returned to a reprocessing unit for medical devices (AEMP). Currently, only manual intraoperative counting of instruments and materials using lists to be processed is known. An automated process according to the present disclosure significantly simplifies quality assurance and may also accelerate the time required for determining intraoperatively lost items, since it is also possible to clearly show which items may not be present. Furthermore, human errors can be avoided.

In yet other words, X-ray inspection technology is crucial. Comparable devices are mainly used to inspect luggage and postal parcels to detect explosives or other illegal substances.

The inspection technology for food is primarily intended to detect foreign bodies such as broken knives or other metals as well as pieces of bone, glass or plastic in the finished packaging. The technology is also used to search for foreign bodies in other products made of plastic, wood or textiles that should not be present in the actual product and to check the products for other features. However, the established technology focuses on the detection of foreign bodies, not on their size or pose. It is state of the art for X-ray inspection devices to be combined with other systems such as scales and optical systems in order to verify other desired product characteristics such as weight and shape. The X-ray inspection devices usually have a conveyor belt that automatically transports the items to be inspected through the scanners. In the case of food packaging, the devices can achieve throughput speeds of 1.5 m/s with an image resolution of 0.4 mm and 150 to 255 shades of gray.

Further aspects are described in more detail below.

It is preferred if a housing surrounds the above components, in particular the capturing apparatus and the medical and/or surgical items to be identified.

It is preferable if two capturing units are provided, which are preferably arranged orthogonally to each other.

It is advantageous if a conveyor belt is provided and arranged between the at least one first scanner unit and the detector so as to transport the medical and/or surgical items on the conveyor belt. The medical and/or surgical items may be isolated, but may also be in a transport container such as a sieve basket and/or sterile container. Furthermore, the conveyor belt preferably protrudes beyond the housing at the inlet and outlet.

Furthermore, it is advantageous if a first scanner unit is arranged in a fixed position orthogonal to the conveyor belt.

In other words, various scanner configurations are provided, for example as a line scanner with a conveyor belt (with corresponding peripherals such as at least one X-ray-impermeable curtain), as an area scanner with and without conveyor belt and variations of the scanner unit number, whereby two scanner units are provided in an orthogonal arrangement or in a variable position, but advantageously only one scanner unit is provided in a fixed position in the orthogonal scanning direction to the conveyor belt. A line scanner is a scanner whose core element is a CCD line that is transported over an item in microsteps by a stepper motor and is thus scanned line by line. In contrast, the area scanner scans an area.

It is preferable if a curtain impermeable to X-rays is provided, which is arranged in such a way as to shield the at least one scanner unit as well as the medical and/or surgical items from the environment during identification. The X-ray radiation is shielded from the environment by curtains over the conveyor belt made of appropriate material.

It is advantageous if the representation of the medical and/or surgical items is performed by the imaging and computing unit in real time. In other words, a monitor is used to display the passing goods/medical and/or surgical items in real time.

Preferably, a sorting unit is provided, which is provided and configured to automatically remove and/or separate the medical and/or surgical items according to their current pose and to feed them to a subsequent process. In other words, it is provided that the present disclosure can also be used in the AEMP. Here, the image data can be used to create a spatial model of all chaotically arranged instruments in the cluster, which can be used by a robotic unit to automatically remove or separate the items/instruments according to their current pose and to feed them to subsequent processes.

In addition, the present device may also have an integrated automatic ejection mechanism for non-compliant goods/compliant medical and/or surgical items.

It is preferred if the imaging and computing unit is provided and configured to access a corresponding item list via an interface for matching the identified medical and/or surgical items and to match the identified medical and/or surgical items with the item list. In other words, an interface can access and match corresponding instrument and/or material lists.

It is advantageous if an artificial intelligence device facilitates identification based on previously generated 3D data of the medical and/or surgical items. In other words, with the help of previously created 3D data of medical and/or surgical items by an AI, an identification in particular only by a view through an X-ray-based imaging method (i.e. usually by the top view of the longest axes of the instruments) should be sufficient. Identification is carried out by matching previously learned X-ray-based images, which can be performed with a very high reproducibility compared to optical systems due to the elimination of external disturbance variables such as changes in light intensity, reflections, coloring, etc. It is proposed that the identification of the medical and/or surgical items may take place not only at article number level, but also at batch level, if corresponding differences in geometry, for example caused by shape and pose tolerances of the individual medical and/or surgical items, enable a clear assignment.

Furthermore, the present disclosure relates to an identification system comprising a number, in particular plurality, of medical and/or surgical items, a capturing apparatus comprising or using at least one scanner unit and a detector, and an imaging and computing unit, preferably arranged on/in a housing, wherein the at least one scanner unit is or has a source of electromagnetic radiation, preferably X-rays, and the imaging and computing unit is provided and configured to recognize and output the completeness of the medical and/or surgical items and/or the absence of at least one medical and/or surgical item, preferably postoperatively and/or intraoperatively.

In particular, it is preferable if the system communicates with other systems such as inventory management program systems. The software can be used to notify other stations in the AEMP in advance if instruments cannot be found and need to be replaced. At present, a loss of instruments is only detected when the packing lists are processed during loading of the sieve baskets in the AEMP, i.e. just-in-time provision of goods can be carried out with the proposed system.

Furthermore, it is possible to check for damage to instruments, e.g. defects in cutting edge geometries or internal mechanical components, such as those found in torque limiting systems. These defects may preferably be displayed automatically.

In particular, it is preferred if all items to be identified are located in a sealed container that is permeable to X-rays, but not necessarily to visible light. In particular, the container should automatically pass through the X-ray inspection device and exchange the aforementioned information with peripheral devices via the interface. This minimizes the risk of injury to the user, since the instruments do not have to be handled again for the counting check.

Furthermore, the 3D model of the items/instruments is to be used by an AI to determine whether instruments are inserted into each other and a strategy for problem-free removal is to be determined accordingly. With the help of X-ray imaging methods, a representation is possible here that is no longer possible with optical light as the number of instruments increases.

In all of the above cases, an enclosure/a housing or housing with curtain/curtains is provided for the X-ray source, the sensors and the container and items/instruments to be inspected.

Ideally, the parameters of the X-ray imaging components are configured for use with a sterile container and corresponding sieves. Corresponding software-side filters for imaging are provided.

### BRIEF DESCRIPTION OF THE DRAWINGS

FIG. 1 is a representation illustrating a device or system structure according to a first embodiment of the present disclosure;

FIG. 2 is a representation illustrating a device or system structure according to a second embodiment of the present disclosure; and

FIG. 3 is a representation illustrating the communication between the components.

### DETAILED DESCRIPTION

Configuration examples of the present disclosure are described below on the basis of the associated Figures.

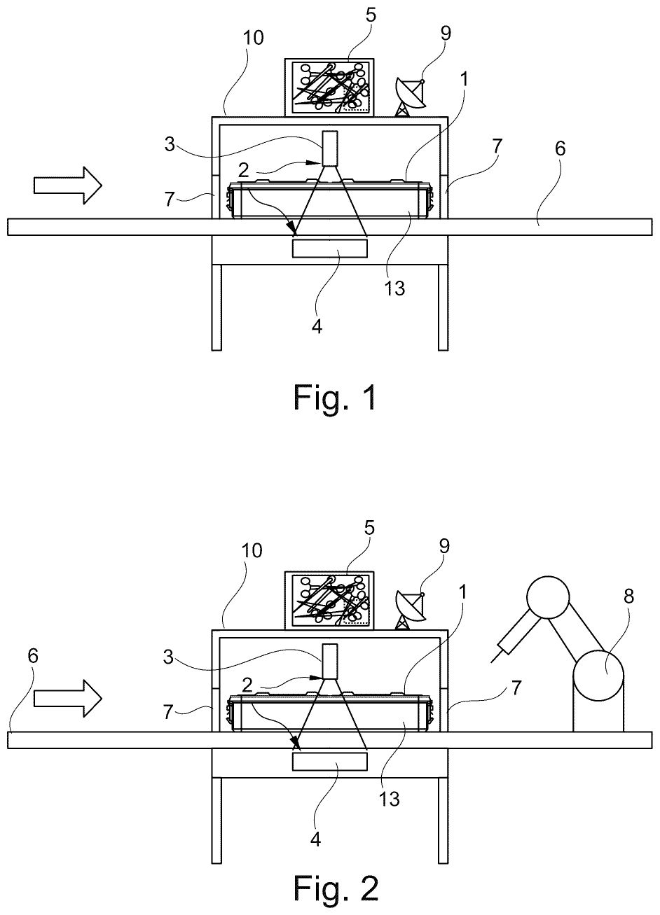

FIG. 1 is a representation illustrating a device or system structure according to a first embodiment of the present disclosure. The device or system for automatically identifying medical and/or surgical items 1 has a capturing apparatus 2. The capturing apparatus 2 has at least a scanner unit 3 and a detector 4. In order to be able to carry out (automatic) identification, the medical and/or surgical items 1 have to be located between the scanner unit 3 and the detector 4 at least briefly—during automatic identification. Furthermore, the device has an imaging and computing unit 5 preferably arranged on/in a housing 10. The imaging and computing unit 5 has a monitor on which the representation of a corresponding image can be displayed.

The at least one scanner unit 3 is a source of electromagnetic radiation, preferably X-rays. The imaging and computing unit 5 is provided and configured to recognize and output the completeness of the medical and/or surgical items 1 and/or the absence of at least one medical and/or surgical item 1, preferably postoperatively and/or intraoperatively.

Furthermore, in FIG. 1, a conveyor belt 6 is provided and arranged between the scanner unit 3 and the detector 4 such that the medical and/or surgical items 1 are transported on the conveyor belt 6. It is preferable for the scanner unit 3 to be arranged in a fixed position orthogonal to the conveyor belt 2. In other words, the detector 4 is provided below the conveyor belt 6, preferably in the center of the housing 10. The scanner unit 3 is arranged inside the housing 10 above the conveyor belt 6. The X-ray source of the scanner unit 3 is aligned in the direction of the detector 4 and is preferably arranged in the center of the detector 4.

By moving the conveyor belt 6 in the direction of the arrow shown in FIG. 1, the medical and/or surgical items 1 are transported between the scanner unit 3 and the detector 4. In this way, all items on the conveyor belt 6, in particular surgical materials and surgical instruments, can be displayed in real time on the monitor of the imaging and computing unit 5.

The medical and/or surgical items 1 are preferably provided in a sieve basket and/or sterile container 13 or another container permeable to electromagnetic radiation, preferably X-rays.

A curtain 7 impermeable to X-rays is arranged to shield the at least one scanner unit 3 as well as the medical and/or surgical items 1 from the environment during identification. The curtain 7 is provided on the housing 20 on the side of the inlet and outlet of the conveyor belt 6. The sides of the housing 10, which extend, at least in sections, parallel to the conveyor belt 6, also serve as shielding.

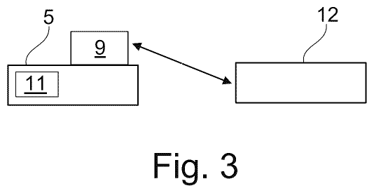

Furthermore, an interface 9 is provided on the housing 10. The imaging and computing unit 5 is provided and configured to access a corresponding item list, preferably stored on a peripheral device 12 (see FIG. 3), via an interface 9 for matching the identified medical and/or surgical items 1 and to match the identified medical and/or surgical items 1 with this list.

FIG. 2 is a representation illustrating a device or a system structure according to a second embodiment of the present disclosure. The second embodiment corresponds to the first embodiment with the difference that the second embodiment is provided for a reprocessing unit for medical devices (AEMP). For this reason, a sorting unit 8 is provided.

The sorting unit 8 is provided at one end (in the direction of the arrow) of the conveyor belt 6. Furthermore, the sorting unit 8 is provided and configured to automatically remove and/or separate the medical and/or surgical items 1 according to their current pose and to feed them to a subsequent process. It is preferred that the sorting unit 8 can communicate with the imaging and computing unit 5 in order to initiate a corresponding intervention by the imaging and computing unit 5.

FIG. 3 is a representation illustrating the communication between the components. The device or system comprises an artificial intelligence device 11 provided and configured to facilitate identification and matching based on previously generated 3D data of the medical and/or surgical items 1. The artificial intelligence device 11 is preferably a part of the imaging and computing unit 5.

The provided communication with a peripheral device 12 takes place via the interface 9, preferably via Bluetooth, radio or other common transmission technologies.

### REFERENCE SIGNS

1 medical and/or surgical items
2 capturing apparatus
3 scanner unit
4 detector
5 imaging and computing unit
6 conveyor belt
7 curtain
8 sorting unit
9 interface
10 housing
11 artificial intelligence device
12 peripheral device
13 sieve basket/sterile container

1. A device for automatically identifying medical and/or surgical items, the device comprising:
a capturing apparatus; and
an imaging and computing unit,
the capturing apparatus comprising and/or using at least one scanner unit and a detector in such a way that medical and/or surgical items are at least briefly located between the at least one scanner unit and the detector during an automatic identification,
the at least one scanner unit is or has a source of X-rays, and
the imaging and computing unit is provided and configured to recognize and output a completeness of the medical and/or surgical items and/or an absence of at least one of the medical and/or surgical items.

2. The device according to claim 1, wherein the capturing apparatus comprises two capturing units arranged orthogonally to each other.

3. The device according to claim 1, further comprising a conveyor belt arranged between the at least one scanner unit and the detector so as to transport the medical and/or surgical items on the conveyor belt.

4. The device according to claim 3, wherein the at least one scanner unit is arranged in a fixed position orthogonal to the conveyor belt.

5. The device according to claim 1, further comprising a curtain that is impermeable to X-rays, the curtain being arranged in such a way as to shield the at least one scanner unit as well as the medical and/or surgical items from a surrounding environment during identification.

6. The device according to claim 1, wherein representation of the medical and/or surgical items is performed by the imaging and computing unit in real time.

7. The device according to claim 1, further comprising a sorting unit configured to automatically remove and/or separate the medical and/or surgical items according to current pose and to feed the medical and/or surgical items to a subsequent process.

8. The device according to claim 1, wherein the imaging and computing unit is configured to access a corresponding item list via an interface for matching the medical and/or surgical items and to match the medical and/or surgical items with the corresponding item list.

9. The device according to claim 1, further comprising an artificial intelligence device configured to facilitate identification and matching based on previously generated 3D data of the medical and/or surgical items.

10. An identification system, comprising:
a plurality of medical and/or surgical items;
a capturing apparatus comprising at least one scanner unit and a detector; and
an imaging and computing unit,
the at least one scanner unit comprising an X-ray source, and
the imaging and computing unit being configured to recognize and output a completeness of the plurality of medical and/or surgical items and/or an absence of at least one of the plurality of medical and/or surgical items.

11. A device for automatically and simultaneously identifying a plurality of medical and/or surgical items, the device comprising:
a capturing apparatus; and
an imaging and computing unit,
the capturing apparatus comprising and/or using at least one scanner unit and a detector in such a way that said plurality of medical and/or surgical items are at least briefly located between the at least one scanner unit and the detector during an automatic and simultaneous identification,
the at least one scanner unit is or has a source of X-rays, and
the imaging and computing unit is configured to recognize and output a completeness of said plurality of medical and/or surgical items and/or an absence of at least one of the plurality of medical and/or surgical items.
